(19) United States
(12) Patent Application Publication
(10) Pub. No.: US 2011/0044970 A1
(43) Pub. Date: Feb. 24, 2011

(54) PHARMACEUTICAL COMPOSITIONS AND METHODS FOR THE TREATMENT OF DRY EYE

(75) Inventor: Gerald Horn, Deerfield, IL (US)

Correspondence Address: WOOD, PHILLIPS, KATZ, CLARK & MORTIMER, 500 W. MADISON STREET, SUITE 3800, CHICAGO, IL, 60661, US

(73) Assignee: Alpha Synergy Development, Inc.

(21) Appl. No.: 12/546,507

(22) Filed: Aug. 24, 2009

(51) Int. Cl. A61K 38/48; A61K 38/18; A61P 27/02

(52) U.S. Cl. 424/94.64; 514/8.1; 514/9.6; 514/14.9; 514/20.8

(57) ABSTRACT

The invention generally relates to methods and compositions for treating dry eye and related conditions by administering compositions comprising compounds that increase capillary permeability of either the lacrimal gland, accessory lacrimal gland, or ocular surface.

### BACKGROUND OF THE INVENTION

Dry eye, also known as Keratoconjunctivitis Sicca (“KCS”), is a condition in which the quality and/or quantity of tears bathing the eye decline. People who have dry eye may experience inflammation, dryness and/or foreign body sensation in the conjunctival region of the eye, light sensitivity, itching, burning or stinging, grittiness, tired eyes, contact lens intolerance, and blurring of vision. Almost all dry eye disorders a result of a loss of water from the tear film. The loss of water from the tear film may be caused by a decrease in tear production and/or an increase in evaporation of tears, which may be a result of an abnormality in mucin or lipid components of the tear film. These phenomena may occur together, but both typically result in increased osmolarity from the normal limit of 311 mOsm/L and may ultimately lead to a decrease in goblet-cell density. A decrease in goblet-cell density affects the production of mucus, which is the major lubricant in the tear film. This aggravates and/or causes inflammation by T-cell activation, causing inflammatory cytokines to be released.

The exposed surface of the globe (eyeball) is kept moist by the tears secreted by the lacrima apparatus, together with the mucous and oily secretions of the other secretory organs and cells of the lids and conjunctiva. The conjunctiva includes a layer covering the protective sclera and episclera, known as the bulbar conjunctiva, and a layer covering the inner eyelids, including the cartilaginous tarsal plate of the lower half of the lids, known as the palpebral conjunctiva. The entire conjunctiva may be referred to as “ocular surface conjunctiva”. The secretion produces the precorneal film, which consists of an inner layer of mucus, a middle layer of lacrimal secretion, and an outer oily film that reduces the rate of evaporation of the underlying watery layer. The normal daily (24-hour) rate of secretion has been estimated at about 0.75 to 1.1 grams (0.03-0.04 ounce avoirdupois). In addition, secretion tends to decrease with age. Chemical analysis of the tears reveals a typical body fluid with a salt concentration similar to that of blood plasma. One of the tear components is lysozyme, an enzyme that has bactericidal action by virtue of its power of dissolving away the outer coats of many bacteria.

The lacrimal glands are paired almond-shaped glands, one for each eye, that secrete the aqueous layer of the tear film. They are situated in the upper outer portion of each orbit. Each lacrimal gland is divided into orbital and palpebral portions by the aponeurosis of the Levator palpebrae superioris muscle, wherein the portions are continuous with each other. The orbital portion is the largest of the portions, and its convex superior surface is lodged in the lacrimal fossa of the orbit. The inferior surface of the smaller palpebral portion lies close to the eye.

The orbital portion contains fine interlobular ducts that unite to form three to five main excretory ducts, joining five to seven ducts in the palpebral portion before the secreted fluid may enter on the surface of the eye. The secreted tears collect in the formix conjunctiva of the upper lid and pass over the eye surface to the lacrimal puncta. The lacrimal gland is a compound tubuloacinar gland. It is made up of many lobules separated by connective tissue, each lobule contains many acini. Each acinus consists of a grape-like mass of lacrimal gland cells, with their apices pointed to a central lumen. The central lumen of many of the units united to form intralobular ducts, and then unite to from interlobular ducts. The lacrimal artery supplies the lacrimal gland. Venous blood returns via the superior ophthalmic vein.

For producing tears, it is necessary to deliver adequate blood supply to the lacrimal glands, which serve through their acinar cells to secrete the aqueous component of tears. Improving the blood supply to these glands may improve the delivery of the aqueous component by increasing the rate of filtration through the lacrimal gland. In the absence of improved blood supply, increased capillary permeability may increase the filtration rate through these glands as well.

All of the three components of tear film (lipid layer, aqueous layer, and mucin layer) offer important physiologic benefits to normal tear function. However, the aqueous layer provides the key nutritional and protective electrolytes, proteins, and nutritional components. Thus, the depletion and/or increase of the aqueous layer most dramatically affects normal tear function.

It has also been shown that patients with chronic dry eye experience increased activation of T-cells. These T-cells release cytokines that may result in: (1) neural signals to the lacrimal gland that disrupt production of natural tears leading to a decrease tear production; (2) tissue damage in the lacrimal glands and/or ocular surface; (3) recruitment of additional T-cells; and/or (4) increased inflammatory cytokine production.

Conditions that may give rise to dry eye include, but are not limited to, Sjogren syndrome, blepharitis, meibomian gland disorder, HIV, herpes zoster, autoimmune disease, the natural aging process, diabetes, long-term contact lens wear, dry environment, surgery that involves corneal incisions or ablates corneal nerves, medications, decreased blinking, eyelids that cannot be closed, pregnancy, polycystic ovary syndrome, acne rosacea, lupus, scleradenma, sarcoidosis, Stevens-Johnson syndrome, Parkinson's disease, smoking, radiation therapy, vitamin A deficiency, and menopause. This wide divergence in causative factors makes it particularly difficult to fashion a successful treatment for dry eye.

There are several techniques for diagnosing and evaluating the severity of a patient's dry eye, including the Ocular Surface Disease Index (OSDI) questionnaire, Tear Break-up Time, tear staining, tear film height, and the Schirmer Test. See Milder, B, The Lacrimal System, Appleton-Century-Crofts, Chapter 8, 1993 and Schirmer, O Studien Zur Physiologie and Pathologie der Tranenabsonderdung and Tranenabfuh, Arch kiln ophthalmol, 1903; 56:197-291, each of which is herein incorporated by reference in its entirety including any references cited therein. Each test provides different information about the tear film of a patient.

The patient's subjective evaluation of the severity of the symptoms can be recorded using the standardized OSDI questionnaire. This subjective evaluation can be confirmed by objective indicators such as the Tear Break-up Time (TBUT) test, and the Schirmer Test. The TBUT test measures the time required for the three-layer tear film to break up. A shortened TBUT test time indicates a decreased quality of tears and is indicative of dry eye. See, Lemp et al., Factors Affecting Tear Film Break Up in Normal Eyes, Arch Ophthalmol 1973; 89:103-105, which is herein incorporated by reference in its entirety including any references cited therein. The Schirmer Test measures the volume of tears produced, and is performed by of placing a small strip of filter paper inside the lower eyelid (conjunctival sac) of each eye for several minutes, allowing tear fluid to be drawn into the filter paper by capillary action. The paper is then removed and the amount of moisture is measured in millimeters. Typically, a measurement of less than 5 mm indicates dry eye. Schirmer, 0 Studien zur physiologie and pathologie der tranenabsonderdung and tranenabfuh, Arch kiln ophthalmol, 1903; 56:197-291.

Despite availability of numerous aqueous solutions for topical treatment of dry eye, the lack of key factors found in physiologic tears that contribute to maintenance of corneal epithelial integrity renders these solutions as inadequate physiologic substitutes.

The only clinically documented means of improving tear production involves the topical application of cyclosporine. Through suppression of T-cells and/or inflammation related intermediary molecules some improvement in tear production may result. However the improvement is only seen in a percentage of patients—typically less than 50%—requires many months before a clinical benefit can be realized, and may be inadequate for the needs of the patient.

Current treatments for dry eye include artificial tears, such as ointments and gels for application to the ocular surface. These provide basic lubrication to the eye surface. Restasis® eye drops (cyclosporine in a castor oil base) are said to help the eyes increase tear production. Other treatments include temporary and permanent punctal occlusions, topical androgen eye drops, topical antibiotics, and oral therapy with polyunsaturated fatty acids.

Accordingly, there is a need for new and improved treatments of dry eye.

### BRIEF SUMMARY OF THE INVENTION

The present invention is generally related to compositions and methods for treating dry eye. One of the key discoveries of the present invention is that increasing capillary permeability of either the lacrimal gland, accessory lacrimal gland, or ocular surface allows increasing effective tear secretion, thus providing a new method of treating dry eye.

In one embodiment, the invention provides a pharmaceutical composition formulated for the treatment of dry eye, wherein said pharmaceutical composition comprises a compound which increases capillary permeability of one or more of the lacrimal gland, accessory lacrimal gland, and ocular surface.

In a preferred embodiment, the compounds that increase capillary permeability is selected from the group consisting of thrombin, semaphorin-A, and an endothelial growth factor. One of the endothelial growth factors suitable for the purposes of the present invention is vascular endothelial growth factor (VEGF-165b).

Other compounds that may be used for the purposes of the present invention generally include any compounds that induce release of serum or serum components in the eye, thus treating dry eye. Generally, any compounds or compositions that release or induce release of endothelial growth factors may be used for the purposes of the present invention.

Some of the suitable compounds include: 1) vasodilators, 2) spasmolytics, 3) (3-2 adrenergic receptor agonists, 4) glucans, 5) glycerols, 6) leukotrienes, 7) fatty acid oxidases, 8) eiconosaids (e.g., 14, 15-epoxyeicosatrienoic acid), 9) tachykinin (inflammatory) peptides (e.g., SP, NKA, NKB, and derivatives), 10) nonangiogenic VEGF family peptides (e.g., VEGF165b, VEGF121); 11) a combination of angiogenic VEGF family peptides with antiangiogenic inhibitors (e.g., a combination of VEGF121 and/or VEGF165 with myristoylated protein kinase C (PKC) inhibitors, such as calyculin a); 12) PKC inhibitors and/or agonists (e.g., such as myristoylated pkc inhibitors, such as N-myristoylation of the PKC pseudosubstrate nonapeptide Phe-Ala-Arg-Lys-Gly-Ala-Leu-Arg-Gln (myr-psi PKC), calyculin A, etc.); 13) specific capillary permeability increasing peptides (e.g., junctional adherence microadhesion molecules jam-a; jam-b; jam-c; jam-d; jam-e, etc.; poly-L-Arg hcl; small GTPases; active PAK, RAC); 14) calcium flux inducers (ionomycin, thapsigargin); 15) thrombin and thrombin derivatives (e.g., thromboxane 2a and thromboxane analogue U46619); 16) neuropilins (e.g., Semaphorin 3A).

The invention also provides methods for the treatment of dry eye comprising administering to a patient in need thereof pharmaceutical compositions of the present invention.

In one embodiment, the invention provides a method of treating dry eye comprising administering to a patient in need thereof a pharmaceutical composition formulated for the treatment of dry eye comprising a compound that increases capillary permeability of one or more of the lacrimal gland, accessory lacrimal gland, and ocular surface.

Other compounds that may be suitable for the purposes of the present invention include salbutamol, bitolterol mesylate, isoproterenol, levalbuterol, metaproterenol, salmeterol, terbutaline, clenbuterol, ritodrine, glyceryl trinitrate, isosorbide mononitrate, isosorbide dinitrate, pentaerythritol tetranitrate, sodium nitroprusside, ildenafil, tadalafil, ovardenafil, and pharmaceutically acceptable salts and derivatives thereof.

In one embodiment, the compounds suitable for the purposes of the present invention trigger VEGF-receptor 1 and/or VEGF-receptor 2 pathways in either the lacrimal gland, accessory lacrimal gland, or ocular surface.

In another embodiment, the compounds have cytokine and/or angiogenic activity.

In another embodiment, the compounds reduce the integrity of adherins junctions.

In a preferred embodiment, the compound is a thrombin or a semaphorin. In the most preferred embodiment, the compound is semaphorin 3-a (sema 3A).

Preferably, the concentration of semaphorin is between 0.1 μg/ml and about 1000 μg/ml, more preferably between 1 μg/ml and about 500 μg/ml, even more preferably between 10 μg/ml and about 200 μg/ml, and most preferably between about 20 μg/ml and about 100 μg/ml.

In one embodiment, the invention provides a method for the treatment of dry eye comprising administering to a patient in need thereof thrombin at a concentration of about 10 NIH units/ml, wherein at least about 1-3 drops of said thrombin is administered to an eye of said patient.

In another embodiment, the invention provides a method for the treatment of dry eye comprising administering to a patient in need thereof semaphorin at a concentration of about 100 μg/ml, wherein at least about 1-3 drops of said semaphorin is administered to an eye of said patient.

In some embodiments, the methods of the present invention include further administering to a patient in need thereof a therapeutically effective amount of an antiangiogenic factor. Generally, antiangiogenic factors selectively inhibit mitogenic endothelial cell activity and/or increase transmembrane tumor necrosis factor in endothelial cell walls.

In some embodiments, the antiangiogenic factor is selected from the group consisting of a myristoylated protein kinase C inhibitor, cycloheximide, VEGF165b, bactericidal/permeability protein, 4-fluoro-5-{[6-methoxy-7-(2-methoxyethoxy) cinnolin-4-yl]amino}-2-methylphenol (VTKI), VEGF-2 tyrosine kinase inhibitors, alemzutab, pegaptanib sodium, ranibizumab, bevacizumab, anecortave acetate, sulfonamide, and pharmaceutically acceptable salts and derivatives thereof.

The antiangiogenic factor and the compound which induces capillary permeability may be formulated in a single pharmaceutical composition or in separate pharmaceutical compositions.

In some embodiments, the methods of the present invention further include administering to the patient an effective amount of soluble tumor necrosis factor.

In certain embodiments, the methods of the present invention further include administering to a patient an effective amount of diacylglycerol (DAG) or its derivatives. In a preferred embodiment, the effective amount of DAG or its derivative is between about 10 μM and about 500 μM.

In some embodiments, the methods of the present invention further include administering to a patient between about 10 mM and about 40 mM of glucose to enhance the effectiveness of the compounds in increasing capillary permeability.

### DETAILED DESCRIPTION OF THE INVENTION

### Definitions

For purposes of the present invention, the terms below are defined as follows.

The term “pharmaceutically acceptable salts” refers to salts of active compounds which are prepared with relatively nontoxic acids. Acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic; propionic; isobutyric; maleic; malonic; benzoic; succinic; suberic; fumaric; mandelic; phthalic; benzenesulfonic; toluenesulfonic, including p-toluenesulfonic, m-toluenesulfonic, and o-toluenesulfonic; citric; tartaric; methanesulfonic; and the like. Also included are salts of amino acids such as arginate and the like, and salts of organic acids like glucuronic or galactunoric acids and the like (see, for example, Berge et al. J. Pharm. Sci. 66:1-19 (1977)).

The term “compound that increases capillary permeability” includes compounds that act in one or more of the following ways: a) increase vasodilation as their primary mechanism of action with secondary permeability increase; b) affect capillaries directly and increase their permeability through anatomic changes in the endothelial cell wall, and c) increase capillary filtration pressure, such as by constricting venules.

The term “vasodilator” refers to compounds that increase vasodilation, usually by relaxing the smooth muscle in blood vessels.

The term “treating” refers to reversing, alleviating, inhibiting, or slowing the progress of the disease, disorder, or condition to which such term applies, or one or more symptoms of such disease, disorder, or condition.

The term “therapeutically effective amount” refers to the amount of the compound that will elicit the biological or medical response of a tissue, system, animal or human that is being sought by the researcher, veterinarian, medical doctor or other clinician or that is sufficient to prevent development of or alleviate to some extent one or more of the symptoms of the disease being treated.

The term “dry eye” includes dry eye and dry eye syndromes, such as Sjogren's syndrome.

### Pharmaceutical Composition and Methods of the Present Invention

The present invention is generally related to compositions and methods for treating dry eye. It allows to achieve one or more of the following: a) improving the chemistry of tears by stimulating the release of endothelial growth factors and b) increasing the volume of tears. In addition, in some embodiments of the invention, these benefits are achieved without utilizing complex chemical compounds. Rather, natural compounds such as thrombin, semaphorin, and endothelial growth factors are preferred.

In one embodiment, the invention provides a pharmaceutical composition formulated for the treatment of dry eye, wherein said pharmaceutical composition comprises a compound which increases capillary permeability of one or more of the lacrimal gland, accessory lacrimal gland, and ocular surface.

In a preferred embodiment, the compounds that increase capillary permeability is selected from the group consisting of thrombin, semaphorin-A, and an endothelial growth factor. One of the endothelial growth factors suitable for the purposes of the present invention is vascular endothelial growth factor (VEGF).

Other compounds that may be used for the purposes of the present invention generally include any compounds that induce release of serum or serum components in the eye, thus treating dry eye. Generally, any compounds or compositions that release or induce release of endothelial growth factors may be used for the purposes of the present invention.

Some of the suitable compounds include: 1) vasodilators, 2) spasmolytics, 3) (3-2 adrenergic receptor agonists, 4) glucans, 5) glycerols, 6) leukotrienes, 7) fatty acid oxidases, 8) eiconosaids (e.g., 14, 15-epoxyeicosatrienoic acid), 9) tachykinin (inflammatory) peptides (e.g., SP, NKA, NKB, and derivatives), 10) nonangiogenic VEGF family peptides (e.g., VEGF165b, VEGF121); 11) a combination of angiogenic VEGF family peptides with antiangiogenic inhibitors (e.g., a combination of VEGF121 and/or VEGF165 with myristoylated protein kinase C (PKC) inhibitors, such as calyculin a); 12) PKC inhibitors and/or agonists (e.g., such as myristoylated pkc inhibitors, such as N-myristoylation of the PKC pseudosubstrate nonapeptide Phe-Ala-Arg-Lys-Gly-Ala-Leu-Arg-Gln (myr-psi PKC), calyculin A, etc.); 13) specific capillary permeability increasing peptides (e.g., junctional adherence microadhesion molecules jam-a; jam-b; jam-c; jam-d; jam-e, etc.; poly-L-Arg hcl; small GTPases; active PAK, RAC); 14) calcium flux inducers (ionomycin, thapsigargin); 15) thrombin and thrombin derivatives (e.g., thromboxane 2a and thromboxane analogue U46619); 16) neuropilins (e.g., Semaphorin 3A).

The induction of increased tear volume via increased capillary permeability offers immediate substantial relief with a physiologic composition that includes most of the organic molecules found in human tears. Leakage of serum from surface capillaries on the conjunctiva provides immediate dry relief with physiological delivery of transudate that may have some secretory modification by conjunctival cuboidal epithelium, and vary from the composition of serum, with its know therapeutic benefits to the ocular surface and particularly the cornea, to even more closely resembling that of human tears, with some secretory IgA, for example, as the transudate passes through squamous conjunctival epithelium. Serum, however, offers a high concentration of serum IgA with similar immune protective properties.

Compounds capable of increasing surface capillary permeability may be applied topically to the eye, and induce their benefit primarily through permeation through conjunctival capillaries along the ocular surface, or via injection, such as subconjunctivally and or directly into the lacrimal and or accessory lacrimal glands. To minimize any unwanted secondary permeability in scleral or episcleral vessels modifications of compounds to prevent or minimize their absorption into deeper tissues such as sclera may be made, including binding of these active compounds to larger nonabsorbed molecules such as albumin, methylcellulose, or dextran.

Some compounds suitable for the present invention increase vasodilation as their primary mechanism of action, with secondary permeability increase; others affect capillaries directly and increase their permeability through anatomic changes in the endothelial cell wall—and yet others increase capillary filtration pressure, such as by primarily constricting venules.

In a preferred embodiment, the compound that increases capillary permeability is thrombin or its derivatives. It is within a skill in the art to modify the thrombin molecule to further improve its functionality for permeability enhancement without other thrombin derived effects.

### Semaphorins

In one embodiment, the compositions suitable for the purposes of the present invention comprise semaphorins.

Class 3 semaphorins, a family of secreted proteins, are implicated in a variety of biologic functions. They are a class of secreted and membrane proteins originally discovered to act as axonal growth cone guidance molecules. They primarily act as short-range inhibitory signals and signal through multimeric receptor complexes. They are usually cues to deflect axons from inappropriate regions, especially important in neural system development. The major class of proteins that act as their receptors are called plexins.

There are 8 major classes of Semaphorins. The first 7 are ordered by number, from class 1 to class 7. The eighth group is class V. Classes 1 and 2 are found in invertebrates only, while classes 3, 4, 6, and 7 are found in vertebrates only. Class 5 is found in both vertebrates and invertebrates and Class V is specific to viruses. Classes 1 and 6 are considered to be homologues of each other, since they are both membrane bound in vertebrates and invertebrates. The same applies to classes 2 and 3, because they are both secreted proteins specific to phylum.

Each class of semaphorins has many subgroups of different molecules that share similar characteristics. For example, Semaphorin Class 3 ranges from Sema-3A to Sema-3E. Each one of the Class 3 Semaphorins are expressed in different regions of the body during development and whilst some encourage the growth of axons, others inhibit it.

All semaphorins are encompassed by the term “semaphorin” as used in the present application.

Sema-3A potentiation of VEGF vascular permeability, and independent increase of vascular permeability in a preferred embodiment is anticipated in concentrations of 30-100 μg/ml, more generally in a range of 10 μg/mL-500 μg/ml; and may be used for these purposes in concentrations ranging from 0.1 μg/mL-1000 μg/ml.

### VEGF Family

In some embodiments of the present invention, the compositions and methods comprise administering VEGF for the treatment of dry eye.

VEGF is a member of a family of six structurally related proteins that regulate the growth and differentiation of multiple components of the vascular system, especially blood and lymph vessels. The angiogenic effects of the VEGF family are thought to be primarily mediated through the interaction of VEGF with VEGFR-2.

The broad term ‘VEGF’ covers a number of proteins from two families, that result from alternate splicing of mRNA from a single, 8 exon, VEGF gene. The two different families are referred to according to their terminal exon (exon 8) splice site—the proximal splice site (denoted VEGFₓₓₓ) or distal splice site (VEGFₓₓₓb).

All VEGF and VEGF-type proteins are encompassed by the term “VEGF” as used in the present invention.

The invention also provides methods for the treatment of dry eye comprising administering to a patient in need thereof pharmaceutical compositions of the present invention.

In one embodiment, the invention provides a method of treating dry eye comprising administering to a patient in need thereof a pharmaceutical composition formulated for the treatment of dry eye comprising a compound that increases capillary permeability of one or more of the lacrimal gland, accessory lacrimal gland, and ocular surface.

Other compounds that may be suitable for the purposes of the present invention include salbutamol, bitolterol mesylate, isoproterenol, levalbuterol, metaproterenol, salmeterol, terbutaline, clenbuterol, ritodrine, glyceryl trinitrate, isosorbide mononitrate, isosorbide dinitrate, pentaerythritol tetranitrate, sodium nitroprusside, ildenafil, tadalafil, ovardenafil, and pharmaceutically acceptable salts and derivatives thereof.

In one embodiment, the compounds suitable for the purposes of the present invention trigger VEGF-receptor 1 and/or VEGF-receptor 2 pathways in either the lacrimal gland, accessory lacrimal gland, or ocular surface.

In another embodiment, the compounds have cytokine and/or angiogenic activity.

In another embodiment, the compounds reduce the integrity of adherins junctions.

In a preferred embodiment, the compound is a thrombin or a semaphorin. In the most preferred embodiment, the compound is semaphorin 3-a (sema 3A).

Preferably, the concentration of semaphorin is between 0.1 μg/ml and about 1000 μg/ml, more preferably between 1 μg/ml and about 500 μg/ml, even more preferably between 10 μg/ml and about 200 μg/ml, and most preferably between about 20 μg/ml and about 100 μg/ml.

In one embodiment, the invention provides a method for the treatment of dry eye comprising administering to a patient in need thereof thrombin at a concentration of about 10 NIH units/ml, wherein at least about 1-3 drops of said thrombin is administered to an eye of said patient.

In another embodiment, the invention provides a method for the treatment of dry eye comprising administering to a patient in need thereof semaphorin at a concentration of about 100 μg/ml, wherein at least about 1-3 drops of said semaphorin is administered to an eye of said patient.

Other compounds that may be used for the purposes of the present invention include compounds that may induce capillary permeability increase via mechanisms not strictly vasodilator in function (though they may include vasodilation), including the angiogenic cytokines, particularly the VEGF family of compounds, phosphatase inhibitors, thrombin, thromboxanes, neuropilins, calcium, and particularly, calcium membrane flux inducers, a variety of peptides, including tachykinins, econosaids, leukotrienes, and glucans. An increase in physiologic tear production can be achieved by varying the concentrations and utilizing combinations of the permeability inducers according to the present invention.

Primary inducers of capillary permeability include members of the family zo known as cytokines, or inducers thereof. Cytokines are a category of signaling proteins and glycoproteins that, like hormones and neurotransmitters, are used extensively in cellular communication. While hormones are secreted from specific organs to the blood, and neurotransmitters are related to neural activity, the cytokines are a more diverse class of compounds in terms of origin and purpose. They are produced by a wide variety of hematopoietic and non-hematopoietic cell types and can have effects on both nearby cells or throughout the organism, sometimes strongly dependent on the presence of other chemicals. The cytokine family consists mainly of smaller, water-soluble proteins and glycoproteins with a mass of between 8 and 30 kDa.

Angiogenic cytokines, which are cytokines that promote new blood vessel formation, include fibroblast growth factor (FGF), vascular endothelial growth factor (VEGF), hepatocyte growth factor (HGF), placental growth factor, stromal cell-derived factor-1-alpha. These molecules typically increase capillary permeability. Further elucidation of the communication and types of molecules involved in cytokine induced angiogenesis and increased capillary permeability have identified different pathways for the angiogenesis and capillary permeability induction.

In some embodiments, the methods of the present invention include further administering to a patient in need thereof a therapeutically effective amount of an antiangiogenic factor. Generally, antiangiogenic factors selectively inhibit mitogenic endothelial cell activity and/or increase transmembrane tumor necrosis factor in endothelial cell walls.

In some embodiments, the antiangiogenic factor is selected from the group consisting of a myristoylated protein kinase C inhibitor, cycloheximide, VEGF165b, bactericidal/permeability protein, 4-fluoro-5-{[6-methoxy-7-(2-methoxyethoxy) cinnolin-4-yl]amino}-2-methylphenol (VTKI), VEGF-2 tyrosine kinase inhibitors, alemzutab, pegaptanib sodium, ranibizumab, bevacizumab, anecortave acetate, sulfonamide, and pharmaceutically acceptable salts and derivatives thereof.

The antiangiogenic factor and the compound which induces capillary permeability may be formulated in a single pharmaceutical composition or in separate pharmaceutical compositions.

In some embodiments, the methods of the present invention further include administering to the patient an effective amount of soluble tumor necrosis factor.

In certain embodiments, the methods of the present invention further include administering to a patient an effective amount of diacylglycerol (DAG) or its derivatives. In a preferred embodiment, the effective amount of DAG or its derivative is between about 10 μM and about 500 μM.

In some embodiments, the methods of the present invention further include administering to a patient between about 10 mM and about 40 mM of glucose to enhance the effectiveness of the compounds in increasing capillary permeability.

One skilled in the art will appreciate that the means for improving tear function, volume, and physiologic composition taught by the present invention can be applied orally with similar potential for benefit by improving oral secretions and, particularly, treating reduced salivary secretion often found in Sjogren's syndrome. This can be accomplished via oral route of administration with brief retention of a volume of fluid for 30 seconds to a few minutes using a compound selected from those taught by the present invention, including but not limited to thrombin, semaphorin 3a, VEGF 165 b, or combinations thereof. Similarly, secretions within the vaginal cavity may be increased using a suppository or other means of application.

### Compositions

Preferably, the compositions administered according to the present invention will be formulated as solutions, suspensions and other dosage forms for topical administration. Aqueous solutions are generally preferred, based on ease of formulation, as well as a patient's ability to easily administer such compositions by means of instilling one to two drops of the solutions in the affected eyes. However, the compositions may also be suspensions, viscous or semi-viscous gels, or other types of solid or semi-solid compositions. Suspensions may be preferred for cytokine synthesis inhibitors which are sparingly soluble in water.

The compositions administered according to the present invention may also include various other ingredients, including but not limited to surfactants, tonicity agents, buffers, preservatives, co-solvents and viscosity building agents. Various tonicity agents may be employed to adjust the tonicity of the composition, preferably to that of natural tears for ophthalmic compositions. For example, sodium chloride, potassium chloride, magnesium chloride, calcium chloride, dextrose and/or mannitol may be added to the composition to approximate physiological tonicity. Such an amount of tonicity agent will vary, depending on the particular agent to be added. In general, however, the compositions will have a tonicity agent in an amount sufficient to cause the final composition to have an ophthalmically acceptable osmolality (generally about 150-450 mOsm, preferably 250-350 mOsm). An appropriate buffer system (e.g., sodium phosphate, sodium acetate, sodium citrate, sodium borate or boric acid) may be added to the compositions to prevent pH drift under storage conditions. The particular concentration will vary, depending on the agent employed. Preferably, however, the buffer will be chosen to maintain a target pH within the range of pH 6-7.5.

Compositions formulated for the treatment of dry eye-type diseases and disorders may also comprise aqueous carriers designed to provide immediate, short-term relief of dry eye-type conditions. Such carriers can be formulated as a phospholipid carrier or an artificial tears carrier, or mixtures of both. As used herein, “phospholipid carrier” and “artificial tears carrier” refer to aqueous compositions which: (i) comprise one or more phospholipids (in the case of phospholipid carriers) or other compounds, which lubricate, “wet,” approximate the consistency of endogenous tears, aid in natural tear build-up, or otherwise provide temporary relief of dry eye symptoms and conditions upon ocular administration; (ii) are safe; and (iii) provide the appropriate delivery vehicle for the topical administration of an effective amount of one or more of the specified cytokine inhibitors.

Examples or artificial tears compositions useful as artificial tears carriers include, but are not limited to, commercial products, such as Tears Naturale®, Tears Naturale II®, Tears Naturale Free®, and Bion Tears® (Alcon Laboratories, Inc., Fort Worth, Tex.). Examples of phospholipid carrier formulations include those disclosed in U.S. Pat. Nos. 4,804,539 (Guo et al.), 4,883,658 (Holly), 4,914,088 (Glonek), 5,075,104 (Gressel et al.), 5,278,151 (Korb et al.), 5,294,607 (Glonek et al.), 5,371,108 (Korb et al.), 5,578,586 (Gionek et al.); the foregoing patents are incorporated herein by reference to the extent they disclose phospholipid compositions useful as phospholipid carriers of the present invention.

Other compounds designed to lubricate, “wet,” approximate the consistency of endogenous tears, aid in natural tear build-up, or otherwise provide temporary relief of dry eye symptoms and conditions upon ocular administration the eye are known in the art. Such compounds may enhance the viscosity of the composition, and include, but are not limited to: monomeric polyols, such as, glycerol, propylene glycol, ethylene glycol; polymeric polyols, such as, polyethylene glycol, hydroxypropylmethyl cellulose (“HPMC”), carboxy methylcellulose sodium, hydroxy propylcellulose (“HPC”), dextrans, such as, dextran 70; water soluble proteins, such as gelatin; and vinyl polymers, such as polyvinyl alcohol, polyvinylpyrrolidone, povidone and carbomers, such as carbomer 934P, carbomer 941, carbomer 940, carbomer 974P. Other compounds may also be added to the ophthalmic compositions of the present invention to increase the viscosity of the carrier.

Examples of viscosity enhancing agents include, but are not limited to polysaccharides, such as hyaluronic acid and its salts, chondroitin sulfate and its salts, dextrans, various polymers of the cellulose family; vinyl polymers; and acrylic acid polymers. In general, the phospholipid carrier or artificial tears carrier compositions will exhibit a viscosity of 1 to 400 centipoises (“cps”). Topical ophthalmic products are typically packaged in multidose form. Preservatives may be required to prevent microbial contamination during use. Suitable preservatives include benzalkonium chloride, chlorobutanol, benzododecinium bromide, methyl paraben, propyl paraben, phenylethyl alcohol, edetate disodium, sorbic acid, polyquaternium-1, or other agents known to those skilled in the art. Such preservatives are typically employed at a level of from 0.001 to 1.0% w/v. Unit dose compositions of the present invention will be sterile, but typically unpreserved. Such compositions, therefore, generally will not contain preservatives.

The preferred compositions of the present invention are intended for administration to a human patient suffering from dry eye or symptoms of dry eye. Preferably, such compositions will be administered topically. In general, the doses used for the above described purposes will vary, but will be in an effective amount to eliminate or improve dry eye conditions. Generally, one to two drops of such compositions will be administered from once to many times per day.

A contact lens may optionally be used to allow for extravasation of vasoactive substance over a more prolonged time period. Vasoactive substances such as Thrombin and Thromboxane A may further induce increase in tear volume via venular vasoconstriction and increased perfusion through lacrimal, accessory lacrimal and surface microvessels; where increased paracellular endothelial openings that increase capillary permeability can further enhance this benefit.

The invention is illustrated by the following non-limiting examples.

### EXAMPLES

In the Examples below, tear volume was assayed using phenol thread testing, where a fresh phenol thread was inserted in the lateral canthus and allowed to wet for 15 seconds. It was then removed and the length of visibly moistened thread was measured. This test is identical to a Schirmers test, except phenol thread was used instead of Shirmers litmus type paper and the typical measurement time of 15 sec is shorter than the normally used in a Schirmer's test.

### Example 1

### Clinical Benefit Study, Thrombin

It is well known that despite its severity, dry eye syndrome can be difficult to correlate with clinical tear function measurements. Tear volume is but one indicator of tear function. Tear composition, tear break up are others. Tear quality can be an equal or greater factor in therapeutic benefit than tear volume or even break up (an indicator of surfactant effectiveness at keeping tear film in place). Therefore clinical dry eye may or may not easily correlate with any specific tear test, but it will correlate with a spectrum of described symptoms, such as inability to wear contact lenses for prolonged periods of time each day.

Subject had known moderate dry eye, with baseline phenol thread at 15 seconds of 10 mm right eye and 11 mm left eye average. Subject wore extended wear lenses for daily wear only due to dryness, using high O2 permeable, high water content lenses (O2 Optix, Dk=110, 67% lotrafilcon B).

The subject reported considerable difficulty wearing lenses beyond 12 hours, with problems becoming particularly acute at night and in the event of inadvertently falling asleep with lenses in place as occurs periodically. For several years, the subject experienced dramatic vision reduction when sleeping with lenses in for even 30 minutes, and frequently would require muro 5% (hypertonic NaCl solution for removal due to dryness and lens adherence when this occurs. Subject has not slept in lenses overnight for this reason.

Thrombin, 1 NIH unit/ml, was applied topically, 2 gtts ou×2, 1 minute apart. The subject then proceeded to go to sleep with his lenses in place. Subject awoke after 4 hours and reports being able to see the lit red diode on a security system about 10 feet away with perfect clarity, the diode having a very finite crisp point of light appearance, as opposed to the scattered streak effect typically experienced after falling asleep for even short periods of time. Further, subject described total comfort. On arising the next morning the subject had identical vision and comfort, and continued to wear the lenses for another 5 hours before degradation in vision noted. No additional thrombin however was applied after arising.

### Example 2

### Tear Volume Study, Thrombin

Baseline (no cl's) Phenol thread test, 15 sec, lateral canthus:

| | | | |
|---|---|---|---|
| Od | 9.5 mm average | Os | 11.5 mm average |

Two drops of 10 NIH Units/ml were administered to each eye, one min
apart: . = 15 min post instillation

| | | | |
|---|---|---|---|
| Od | 17 mm, 15 mm | Os | 28 mm, 17 mm, 19 mm |

30 min

| | | | |
|---|---|---|---|
| Od | 14 mm, 14 mm | Os | 15 mm, 23 mm, 22 mm |

Reinstallation: 2 gtts q 1.5 min × 3 ou:
15 min post reinstillation

| | | | |
|---|---|---|---|
| Od | 28 mm, 26 mm, 24 mm | Os | 20 mm, 24 mm, 25 mm |

45 min post reinstallation

| | | | |
|---|---|---|---|
| Od | 25 mm, 27 mm, 25 mm | Os | 19 mm, 17 mm, 17 mm |

### Example 3

### Semaphorin 3-A to Treat Dry Eye

Baseline: Artificial tears, 15 min. wait., then Phenol thread, 15 sec,
lateral canthus

| | | | |
|---|---|---|---|
| Od | 12 mm avg | Os | 11.5 mm avg |

101 ug/ml Semaphorin 3-a; 2 gtts ou × 2, 1 min. apart:
15 min. post instillation:

| | | | |
|---|---|---|---|
| Od | 24.5 mm avg | Os | 14.5 mm avg |

30 min

| | | | |
|---|---|---|---|
| Od | 21.5 mm avg | Os | 15 mm avg |

Reinstillation × 3; 1.5 min apart, 2 gtts each application
15 min

| | | | |
|---|---|---|---|
| Od | 15.5 mm avg | Os | 12 mm avg |

30 min

| | | | |
|---|---|---|---|
| Od | 20 mm avg | Os | 26 mm avg |

80 min

| | | | |
|---|---|---|---|
| Od | 17 mm avg | Os | 17.5 mm avg |

### Example 4

### Semaphorin and VEGF to Treat Dry Eye

| Baseline | | | |
|---|---|---|---|
| Od | 11 mm avg | Os | 12 mm os avg. |

Semaphorin 3-a 50 ug/cc 2 gtts ou × 2 1 min apart

| 15 minutes post instillation: | | | |
|---|---|---|---|
| Od | 17 mm avg | Os | 17 mm avg |
| 30 min: | | | |
| Od | 14 mm avg | Os | 15 mm avg. |

Instillation of Vegf 165 b 75 ug/cc:

| 15 min: | | | |
|---|---|---|---|
| OD | 26 mm avg | Os | 23 mm avg, |

### Example 5

O2 Optix 67% lotrafilcon B; high )s, high water content Dk = 110 in place
ou; tear volume measurement (quantifies example 1)
phenol thread, 15 sec measurements:
Baseline

| | | | |
|---|---|---|---|
| od | 15.66 mm avg. | os | 19.00 mm avg. |

Vegf 165 b 80 ug/ml: i gtt q 1 min × 3 ou
20 min post instillation:

| | | | |
|---|---|---|---|
| od | 28 mm avg | os | 31.33 mm avg |

50 min post instillation:

| | | | |
|---|---|---|---|
| od | 19.33 mm avg | os | 22 mm avg |

Slight redness, very mild stinging sensation (0.5/4).
Reinstillatin Thrombin 2 U/ml i gtt ou q 1 min × 3
40 min post instillation:

| | | | |
|---|---|---|---|
| od | 24.33 mm avg. | os | 26.66 mm avg. |

Other:
Much improved contact lens wear comfort after Thrombin: 0/4 stinging.

We claim:

1. A pharmaceutical composition formulated for the treatment of dry eye, wherein said pharmaceutical composition comprises a compound which increases capillary permeability of one or more of the lacrimal gland, accessory lacrimal gland, and ocular surface.

2. The pharmaceutical composition of claim 1, wherein said compound is selected from the group consisting of thrombin, semaphorin-A, and an endothelial growth factor.

3. The pharmaceutical composition of claim 1, wherein said endothelial growth factor is vascular endothelial growth factor VEGF-165b.

4. A pharmaceutical composition formulated for the treatment of dry eye comprising thrombin at a concentration of about 10 NIH units/ml.

5. A pharmaceutical composition formulated for the treatment of dry eye comprising semaphorin 3-A at a concentration of about 100 μg/ml.

6. A method of treating dry eye comprising administering to a patient in need thereof the pharmaceutical composition of claim 1.

7. The method of claim 6, wherein said compound is selected from the group consisting of thrombin, semaphorin 3-A and an endothelial growth factor.

8. The method of claim 6, wherein said compound induces release of protein serum components, wherein said protein serum components improve epithelial integrity.

9. The method of claim 6, wherein said compound induces release of endothelial growth factors in one or more of the lacrimal gland, accessory lacrimal gland, or ocular surface.

10. The method of claim 6, further comprising administering to said patient a therapeutically effective amount of an antiangiogenic factor.

11. The method of claim 10, wherein said antiangiogenic factor is selected from the group consisting of a myristoylated protein kinase C inhibitor, cycloheximide, VEGF165b, bactericidal/permeability protein, 4-fluoro-5-{[6-methoxy-7-(2-methoxyethoxy) cinnolin-4-yl]amino}-2-methylphenol (VTKI), vegf 2 tyrosine kinase inhibitors, alemzutab, pegaptanib sodium, ranibizumab, bevacizumab, anecortave acetate, sulfonamide, and pharmaceutically acceptable salts and derivatives thereof.

12. The method of claim 6, further comprising administering between about 10 μM to about 500 μM of diacylglycerol (DAG) or its derivative to said patient.

13. The method of claim 7, further comprising administering between about 10 mM to about 40 mM of glucose to said patient.

14. The method of claim 7, comprising administering to a patient in need thereof thrombin at a concentration of about 10 NIH units/ml, wherein at least about 1-3 drops of said thrombin is administered to an eye of said patient.

15. The method of claim 7, comprising administering to a patient in need thereof semaphorin at a concentration of about 100 μg/ml, wherein at least about 1-3 drops of said semaphorin is administered to an eye of said patient.
